(19) United States
(12) Patent Application Publication
(10) Pub. No.: US 2011/0151439 A1
(43) Pub. Date: Jun. 23, 2011

(54) SYSTEM AND METHOD FOR THE RAPID IDENTIFICATION OF BIOLOGICAL AND CHEMICAL ANALYTES

(75) Inventor: DIMITRA N. STRATIS-CULLUM, Laurel, MD (US)

(73) Assignee: UNITED STATES GOVERNMENT AS REPRESENTED BY THE SECRETARY OF THE ARMY, Washington, DC (US)

(21) Appl. No.: 12/642,007

(22) Filed: Dec. 18, 2009

(51) Int. Cl. C12Q 1/68; C12M 1/34

(52) U.S. Cl. 435/6; 435/287.2

(57) ABSTRACT

A system and method for the rapid identification of biological and chemical analytes that includes an enzyme substrate compound; an aptamer recognizing an analyte; and a recognition probe that comprises a first terminus operatively coupled to an enzyme catalyzing the enzyme substrate compound; a second terminus operatively coupled to an enzyme inhibitor corresponding to the enzyme, wherein the aptamer is positioned between the first terminus and the second terminus; and a stem loop structure positioned between the first terminus and second terminus is provided.

### GOVERNMENT INTEREST

The embodiments described herein may be manufactured, used, and/or licensed by or for the United States Government without the payment of royalties thereon.

### BACKGROUND

1. Technical Field

The embodiments described herein generally relate to methods, tests, and devices that include aptamer-based sensors. More particularly, the embodiments described herein pertain to an apparatus with an aptamer-based probe that uses enzymatic signaling for detection.

2. Description of the Related Art

Generally, an analyte detection sensing system is composed of two parts: a probe to recognize the analyte of interest, and a transducer to convert that information into a measurable signal.

Currently, many analyte detection systems, such as enzyme-linked immunosorbent assay (ELISA) employ an antibody-based probe. With antibody-based probes, an antibody is first immobilized on a substrate surface and then an antigen is introduced to bind to the antibody by a “lock-and-key” mechanism. Typically, another antibody is then introduced (the “detector” antibody) which will also bind to the antigen but possesses a conjugated label. An analyte detection system that includes an antibody-based probe requires many steps to accurately sense the presence of an analyte (e.g., washings, centrifugations, incubations, etc.).

For example, in an ELISA, an antibody is used as a recognition element (or recognition probe) for a specific target of interest. The target binds to this capture antibody, where the capture antibody is immobilized on a solid support, and a subsequent rinsing step is required to remove any unbound sample components. A second antibody is then introduced that also recognizes the target and forms a “sandwich” of two antibodies, both of which are bound to the target. After additional washing steps to remove the unbound secondary antibody, a third enzyme-conjugated antibody is introduced that binds to the second antibody. Similarly, several washing steps are required to remove the non-specifically bound enzyme antibody. Finally a substrate is introduced, and the enzyme reacts with the substrate to produce a measurable product.

As a consequence of these numerous steps, an analyte detection system with an antibody-based probe is very time-consuming. For example, a single assay typically requiring an entire day for completion. Moreover, the cost and availability of all the materials required for each assay represent other obstacles for integration of these sensors in a time-sensitive environment.

In another field, current aptamer-based sensing systems may provide homogenous and rapid sensing of the presence of a molecular switch (e.g., a molecule with a pronounced “on” and “off” state) for a single step recognition and signaling, known as a molecular aptamer beacon (MAB). MABs are aptamer-based probes that bind to specific nucleic acids in homogenous solutions. By design, MABs are hairpin shaped molecules with an internally quenched fluorophore whose fluorescence is restored when they bind to a target nucleic acid sequence. Since MABs rely on fluorescence for signaling, MABs suffer from high false negatives and false positives rates due to environmental interferences.

Although other configurations of the signaling aptamers exist, what is common in each case is a conformational change in the aptamer upon binding to cause a change in the observed signal when probing the attached molecules on one or both ends. Signaling aptamers, regardless of their configuration, are currently limited by the same shortcomings as MABs—namely, high false negative and false positive rates due to environmental interferences.

### SUMMARY

In view of the foregoing, an embodiment herein provides an aptamer probe system comprising an enzyme substrate compound; an aptamer recognizing an analyte; and a recognition probe comprising: a first terminus operatively coupled to an enzyme catalyzing the enzyme substrate compound; a second terminus operatively coupled to an enzyme inhibitor corresponding to the enzyme, wherein the aptamer is positioned between the first terminus and the second terminus; and a stem loop structure positioned between the first terminus and second terminus.

In addition, in such a system, the enzyme inhibitor may prevent the enzyme from catalyzing the enzyme substrate compound. Moreover, after exposure to an analyte that separate the enzyme inhibitor and the enzyme to restore the enzyme to catalyzing the enzyme substrate compound. Additionally, the recognition probe may comprise an anti-thrombin aptamer. Furthermore, the recognition probe may form a G-quartet in the presence of a thrombin protein. In addition, the analyte may comprise any of a protein, a peptide, a peptide nucleic acid, a nucleoside triphosphate, a carbohydrate, a lipid, a virus, a cell fragment, and a whole cell.

Moreover, in such a system, the enzyme may comprise any of nucleases, proteases, and glycosidases. Furthermore, the enzyme may comprise a hydrolase enzyme. The enzyme may also comprise a butyrylcholinesterase, and the analyte comprises a cholinesterase inhibitor. The enzyme substrate compound may comprise any of acetylcholine and butyrylcholine, and the enzyme may comprise any of acetylcholinesterase and butyrylcholinesterase. The enzyme substrate compound may also comprise benzoyl-arginine-ethyl-ester, and the enzyme may comprise papain. The enzyme substrate compound may further comprise urea, and the enzyme may comprise urea aminohydrolase.

The stem loop structure of such a system may comprise a stem comprising a double-stranded region having a length that is greater than six nucleotides. The enzyme inhibitor may comprise a small-molecular phosphoramidite. In addition, the enzyme substrate compound may comprise DABCYL-bAla-ala-Gly-Leu-AlaBAla-EDANDS.

In addition, the embodiments described herein provides an aptamer probe apparatus comprising an aptamer; a recognition probe comprising: a first terminus operatively coupled to an enzyme; and a second terminus operatively coupled to an enzyme inhibitor, wherein the aptamer is positioned between the first terminus and the second terminus; an enzyme substrate compound that becomes any of colorimetric, fluorescent, and electrochemically active when catalyzed by the enzyme; and a structure incorporated into the recognition probe that brings the first terminus and the second terminus within close proximity to each other, wherein the enzyme inhibitor prevents the enzyme from catalyzing the enzyme substrate compound.

Additionally, after exposure to an analyte that is a complement to the aptamer, the aptamer may be structurally altered to sufficiently separate the enzyme inhibitor and the enzyme to restore the enzyme to catalyzing the enzyme substrate compound. Additionally, the enzyme inhibitor may comprise a small-molecular phosphoramidite. The enzyme substrate compound may also comprise DABCYL-bAla-ala-Gly-Leu-AlaBAla-EDANDS.

Moreover, embodiments described herein provide an aptamer probe system comprising an enzyme substrate compound; an aptamer complementing an analyte; and a recognition probe comprising a first terminus operatively coupled to an enzyme catalyzing the enzyme substrate compound and a second terminus operatively coupled to an enzyme inhibitor corresponding to the enzyme, wherein the aptamer is positioned between the first terminus and the second terminus and forms a structure where the enzyme inhibitor, coupled to the second terminus, interacts with the enzyme, coupled to the enzyme to thereby inhibit the enzyme catalyzing the enzyme substrate compound.

Furthermore, embodiments described herein provide a method of detection, the method comprising providing a substrate; providing a recognition probe comprising an aptamer comprising a nucleic acid that binds to a specific, non-nucleic acid target analyte, wherein the recognition probe comprises a first terminus and an oppositely positioned second terminus; operatively connecting the first terminus to an enzyme; operatively connecting the second terminus to an enzyme inhibitor, wherein the enzyme inhibitor inhibits the enzyme from reacting with the substrate; introducing a target to the substrate that is recognized by the recognition probe causing the enzyme and the enzyme inhibitor to instantly become active thereby causing the enzyme to react with the substrate; modifying the substrate based on the reaction between the enzyme and the substrate, wherein the modified substrate comprises any of colorimetric, fluorescent, and electrochemically active properties; and detecting properties of the target based on the modified substrate.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1 illustrates a schematic diagram of an aptamer-based probe, in a neutral state, according to at least one embodiment described herein;

FIG. 2 illustrates a schematic diagram of the aptamer-based probe shown in FIG. 1, in an activated state, according to an embodiment described herein; and

FIG. 3 illustrates a flow diagram according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

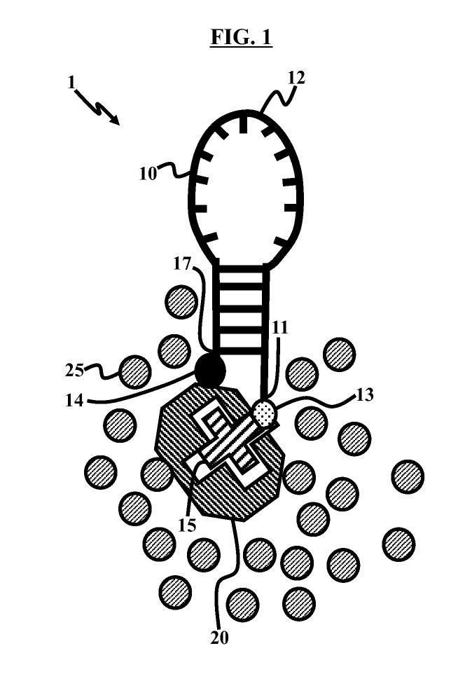

The embodiments described herein provide methods, tests and devices that include aptamer-based analyte detection systems. More particularly, the embodiments described herein provide aptamer-based probes that use enzymatic signaling in an analyte detection system that provides improved measurements in a time-sensitive environment despite environmental interferences. In addition, the various embodiments described herein provide an increase in sensitivity. Referring now to the drawings, and more particularly to FIGS. 1 through 3, where similar reference characters denote corresponding features consistently throughout the figures, there are shown preferred embodiments.

As discussed in further detail below, the embodiments described herein employ aptamer-based probes with enzymatic signaling. In contrast with the antibody-based probe described above, the aptamer-based probes described herein utilize synthetic oligonucleotides (or short strands of approximately 100 bases or less) that bind to specific target molecules based the structure and bonding characteristics of deoxyribonucleic acids (DNA). Synthetic nucleic acid bioreceptors are also known as aptamers. Aptamer-based sensors provide commercially significant advantages over antibody-based biosensors. For example, aptamers may be reused and manipulated multiple times, whereas antibody assays are too fragile and sensitive to be used more than once. Aptamers are also mass-producible, because their production is very reproducible and reliable and do not involve the use of living organisms. In addition, an aptamer-based sensor with single-step recognition of the target material provides a reduction of assay time from a day to minute(s). Consequently, aptamer-based sensors are well suited for time-sensitive environments; e.g., for the soldier in a battlefield, and represents increased survivability through real-time detection of minute amounts of toxic materials.

The embodiments herein utilize enzyme and enzyme inhibitors as being complimentary phenomena to produce a “switching” effect, as described in further detail below. In addition, by manipulating characteristics such as temperature and ionic strength of a buffer solution, the presence of a complement nucleic acid strand amplifies the switching effect; i.e., to turn one phenomena “OFF” and the other “ON”, and vice-versa. The switching effect mentioned above is derived from the distance between an enzyme and its enzyme inhibitor. As described in further detail below, with reference to the accompanying figures, this distance can be modified by an aptamer-based recognition element.

As noted above, adjustments to the temperature and ionic strength of a solution amplifies the described switching effect. At elevated temperatures and with the addition of a target recognized by the aptamer (e.g., a chemical or biological that the aptamer strand has been developed to specifically recognize), equilibrium of some embodiments herein favors the aptamer undergoing a conformation change that alters the spatial configuration of the enzyme and enzyme inhibitor in relation to each other, thereby diminishing the effect of the enzyme inhibitor in the solution and causing the enzyme to produce the greater effect in the substrate solution. In other embodiments, described in further detail below, the enzymatic signaling increases with increasing ionic strength of solution and also with the presence of the target molecule (e.g., a thrombin protein), as ionic media and protein targets can cause DNA to fold into certain specific conformations. For example, when using an anti-thrombin aptamer, the aptamer folds into a G-quartet formation, forcing a separation of an enzyme and its complement enzyme inhibitor and allowing the enzyme to readily react with the substrate solution. The G-quartet is a stable fold of the anti-thrombin oligonucleotide, compared to a non-specific/random fold, as G bases share a very strong bonding energy. Having this sort of “checks and balances” system of measurement provides a more reliable and efficient method for the detection of harmful materials. Consequently, the embodiments described herein provide exemplary performance where rapidity is necessary, such as on the battlefield.

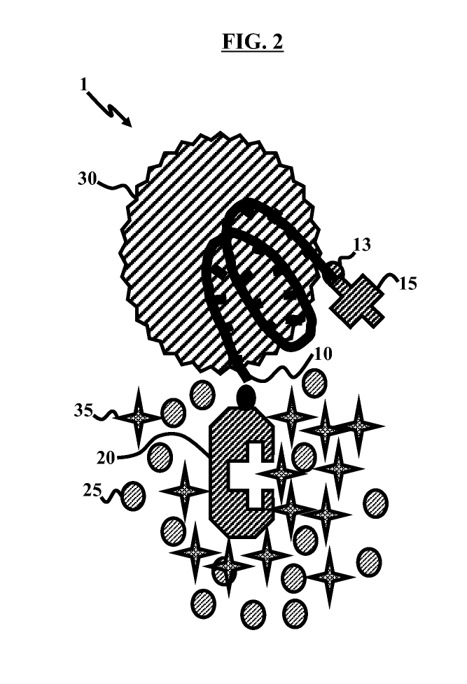

As will be appreciated by one skilled in the art, the embodiments described herein may be embodied as a method, a testing process or apparatus, or a device that utilizes an analyte detection system. A schematic diagram illustrating an aptamer-based probe 1, with a stem-loop and a structure switching design, is provided in FIGS. 1 and 2. FIG. 1 illustrates the probe 1 in a neutral state; i.e., before the aptamer-based probe has been activated, and FIG. 2 illustrates the probe 1 in the activated state. The aptamer-based probe 1, as shown in FIG. 1, includes a recognition probe 10, a stem loop 12, an enzyme attachment 13, an enzyme inhibitor attachment 14, an enzyme 15, an enzyme inhibitor 20, and a substrate 25.

In the embodiment shown in FIG. 1, recognition probe 10 includes an aptamer that is composed of a nucleic acid that is capable of binding to a specific, non-nucleic acid target. Recognition probe 10 is configured to recognize a variety of specific targets including, but not limited to, chemicals, inorganic molecules, organic molecules, biochemicals, proteins, viruses, toxins, whole cells, spores, and so forth. In one example, recognition probe 10 is configured for a specific protein target, for example thrombin, by including an anti-thrombin nucleic acid strand to complement all or a portion of the genetic material for the thrombin protein. Further non-limiting examples are described below.

As noted above, the specific aptamer selected for recognition probe 10 can vary depending on the analyte of interest. In particular, recognition probe 10 is selected such that recognition probe 10 includes a specific nucleic acid sequence that binds to a non-nucleic acid target of interest to allow for the specific recognition. Recognition probe 10 optionally enables specificity down to single base-pair mismatch. For example, it is possible to use previously available and reported aptamer probes, or develop new recognition elements using standard aptamer selection methods. Selection and use of an aptamer for recognition probe 10 allows for the detection of non-nucleic acid targets including a wide range of chemical and biological analytes.

As shown in FIG. 1, one terminus 11 of recognition probe 10 can be attached to enzyme 15, via enzyme attachment 13, using a variety of methods. For example, enzyme attachment 13 may utilize enzyme immobilization techniques. Other known methods of attaching a terminus of a recognition probe 10 to an enzyme 15 may be used. In addition, enzyme 15 catalyzes substrate 25, and thereby alters substrate 25 into reacted substrate 35 (shown in FIG. 2). Reacted substrate 35 can take a variety of forms. For example, the reacted substrate 35 can precipitate, be colorimetric, fluorescent, electrochemically active, and so forth.

In the embodiment shown in FIG. 1, the opposite terminus 17 of recognition probe 10 can be attached to an enzyme inhibitor 20, via enzyme inhibitor attachment 14, using a variety of methods. For example, enzyme inhibitor attachment 14 may utilize enzyme inhibitor immobilization techniques. Other known methods of attaching a terminus of a recognition probe 10 to an enzyme inhibitor 20 may be used. Enzyme inhibitor 20 is the complement of enzyme 15. When enzyme 15 is in close proximity to enzyme inhibitor 20, as shown in FIG. 1, enzyme inhibitor 20 inhibits enzyme 15 from reacting with substrate 25.

In addition to the termini features described above, recognition probe 10 also features stem loop 12. In the embodiments described herein, the stem loop 12 may have a stem comprising a double-stranded region that has a length is greater than three nucleotides, with an optimal length between four to eight nucleotides.

As shown in FIG. 1, stem loop 12 forces the termini 11, 17 of recognition probe 10 to be in close proximity with each other. With enzyme 15 in close proximity to enzyme inhibitor 20, due to stem loop 12, enzyme 15 is prevented from reacting to substrate 20. In FIG. 1, since the ends 11, 17 of the aptamer-based probe 1 are in close proximity and causing inhibition of enzyme 15 in the absence of target 30 (shown in FIG. 2), aptamer-based probe 1 is in a “neutral” (or “off”) state.

As shown in FIG. 2, with reference to FIG. 1, however, upon the introduction of a target 30 that is recognized by recognition probe 10, a conformational change to recognition probe 10 occurs. As a result of the conformational change in recognition probe 10, upon the introduction of target 30, enzyme 15 and enzyme inhibitor 20 physically separate, causing enzyme 15 to instantly become active. Enzyme 15 is thus free to react with a substrate 25 to produce a reacted substrate 35 in a measurable amount. The measurable amount of reacted substrate 35 product can take a variety of forms. For example, reacted substrate 35 can be a precipitate or it can have colorimetric, fluorescent, electrochemically active, etc. properties that are detectable.

Once activated due to the presence of target 30 binding with recognition probe 10, enzyme 15 is no longer inhibited by enzyme inhibitor 20 and can now react with substrate 25 to produce reacted substrate 35 in a measurable amount. As with other enzymatic assay systems, enzyme 15 reacts with substrate 25 at a very high turnover rate. Consequently, a single enzyme 15 can react with thousands of substrate 25 molecules causing an exponential amplification of reacted substrate 35. The exponential amplification of reacted substrate 35 effectively amplifies detection of target 30 when target 30 is introduced into a solution (not shown) containing substrate 25.

Hence, embodiments of aptamer-based probe 1 described herein exists in two different configurations: a neutral form (also referred to as an “off” form) is shown in FIG. 1 and exists in the absence of a target 30 that recognition probe 10 recognizes. As shown in FIG. 1, recognition probe 10 has a prominent stem-loop 12 to force the two termini 11, 17 of recognition probe 10 in close proximity to one another. The second configuration of aptamer-based probe 1 is illustrated in FIG. 2 and shows aptamer-based probe 1 in an activated state (referred to as an “on” form) that is taken when aptamer-based probe 1 is in the presence of target 30 recognized by recognition probe 10. When recognition probe 10 recognizes a target 30, the termini 11, 17 of recognition probe 10 physically separate from each other. Those skilled in the art, however, would recognize that conformations other than the disclosed stem-loop are possible, such as a butterfly conformation, to achieve the “on” state and “off” state described above.

In addition, those skilled in the art would understand that enzyme-inhibitor and enzyme-substrate choices are varied and can be tailored to a variety of transduction schemes and signaling substrates, for example, fluorescence transduction used in ELISA.

The embodiments herein may be implemented in different ways, which are described by the following examples which are not to be construed as limiting the embodiments in scope or spirit to the specific procedures described in them. In the examples that follow, a specific enzyme 15 and enzyme inhibitor 20 system may be described. However, those skilled in the art would understand it is possible to customize the embodiments described herein to a variety of enzyme 15 and enzyme inhibitor 20 systems that could exhibit certain desirable advantages, depending on the detection format employed, sensitivity desired, etc. Each of the examples below includes a complementary enzyme 15 and enzyme inhibitor 20 pair, and the ability of both enzyme 15 and enzyme inhibitor 20 to attach independently (e.g., via enzyme attachment 13 and enzyme inhibitor attachment 14) to an aptamer scaffold (e.g., recognition probe 10), while retaining their respective functions. Preferably, enzyme 15 possesses high catalytic activity to afford rapid rates of signal evolution. The choice of enzyme 15 is directly related to the type of substrate 25 conversion to take place. Thus the choice of enzyme 15 may be a consideration in the detection format employed.

In a first example, an aptamer-based biosensor device may be fabricated and used for the detection of chemical and biological threat agents. One example of a suitable enzyme 15 for aptamer-based probe 1 in this application is Cercus Natural Protease (CNP). Moreover, a small-molecular phosphoramidite inhibitor may be utilized as enzyme inhibitor 20. In addition, CNP may be prepared using E. coli expression.

Specific recognition probes 10 (i.e., single stranded ribo or deoxyribonucleic acid oligonucleotides) can be isolated using Systematic Evolution of Ligands by Exponential Enrichment (SELEX) methods for specific recognition capabilities to chemical and biological threat agents. Recognition probe 10 can also be synthesized using commercialized oligonucleotide synthesis methods. The only requirement for commercial production is that the aptamers are synthesized with a switchable configuration (e.g., stem-loop).

Aptamer-based probe 1 is prepared with a linker such as a C3 linker to serve as a spacer between the oligonucleotide of recognition probe 10 and enzyme inhibitor 20. It is also possible to use a variety of linker modifications, which can be optimized for different enzyme and enzyme inhibitor systems. Enzyme inhibitor 20 can also be conjugated using a nucleic acid and inhibitor conjugate for genomic sensing applications.

Once the recognition probe 10 and enzyme inhibitor 20 conjugate is synthesized, the entire enzymatic signaling aptamer (e.g., aptamer-based probe 1) can be prepared using a solution of CNP in 50 mM Tris, 300 nM NaCL, pH 8, that is added to the conjugate at roughly equivalent molar concentrations. The mixture may sit and react overnight at 4 degrees C. and may be purified by anion exchange chromatography.

In one example, substrate 25 may include DABCYL-bAla-ala-Gly-Leu-AlaBAla-EDANDS and is prepared in two steps by solid and solution phase methods known to those skilled in the art. The measurable reacted substrate 35, and to hence monitor enzymatic activity, the fluorescence of EDANS is measured as the assay proceeds. The excitation and fluorescence emission for EDANS are 350 nm (max), and 490 nm (max), respectively. As a consequence of the proceeding steps, the aptamer-based probe 1 for the bioassay can be been synthesized, as illustrated in FIG. 1.

To detect the chemical or biological threat agent of interest, and cause physical separation of enzyme 15 and enzyme inhibitor 20 (as shown in FIG. 2), a simple mixing with a sample solution (not shown in FIGS. 1 and 2) in the analysis buffer (not shown in FIGS. 1 and 2) may be employed. The sample requirements include solution phase samples, although collection from an aerosol to the solution can be used prior to incubation. The aptamer enzyme reagent (e.g., aptamer-based probe 1), will be mixed to a final concentration in the nM range with a target sample (e.g., target 30). Moreover, sample buffers (not shown in FIGS. 1 and 2) are preferably compatible with the buffer used in SELEX preparation of recognition probe 10 to ensure binding with target 30. Incubation with the sample may be performed for several minutes, followed by the addition of substrate 25 and measurement for several additional minutes.

Substrate 25 reacts with activated enzyme 15 (e.g., separated from enzyme inhibitor 20, upon recognition probe 10 binding with target 30) to produce reacted substrate 35 (e.g., a fluorescent EDANS substrate product). For portable biosensing applications, the system may include a low cost biochip format, and could include microfluidics for reduced reagent logistics. The reagents, once prepared (e.g., as described above), could be integrated into the biochip detection device for field use.

In another example, embodiments described herein may be used in food security and defense. With this example, recognition probe 10 can be developed to recognize targets 30 of concern to the chemical/biological detection community as well as to specific pathogens of concern to food safety that are naturally occurring in the food preparation processes. Moreover, by using aptamer-based probe 1, with enzyme 15 and enzyme inhibitor 20 synthesized, the detection of target 30 can be performed in solution; e.g., food samples can be either swabbed or rinsed to collect samples for analysis. The liquid samples may also be introduced as described in the example above. Measurement of reacted substrate 35 can be accomplished as described above and can also be accomplished in a laboratory setting using microplate reader technologies, equipped with excitation lamp, and optical filters.

The methods above can also be applied to home care diagnostics (e.g., insulin tests diabetes and other illnesses), medical diagnostics, efficacy of vaccination, drug discovery, forensics, and proteomics without significant alteration and without undue experimentation by those skilled in the art.

Furthermore, the embodiments described herein may also be used in non-sensor based applications; e.g., fabrication of electronics and bioelectronics (e.g., enzymatic lithography). When embodiments described herein are utilized for nanofabrication, aptamer-based probe 1 may be attached to a nanotip fabrication device, such as a nano-scale or micro-scale cantilever. In addition, substrate 25 may be customized for the fabrication process being employed. For example, the fabrication process may utilize the interaction between enzyme 15 and substrate 25 to produce reacted substrate 35 such that reacted substrate 35 is a precipitate that is insoluble and localizes at the locus of aptamer-based probe 1 activation. In this example, the activation of the aptamer-based probe 1 may occur through thermal changes in temperature that cause a physical separation of enzyme 15 and enzyme inhibitor 20 due to conformational changes in recognition probe 10. Here the aptamer-based probe 1 does not serve as a sensor, but rather as a switching scaffold to be leveraged for nanofabrication/lithography applications.

While not shown in FIGS. 1 and 2, the recognition probe 10 in aptamer-based probe 1 can be embodied in various configurations as is typical in an analyte detection system. Such a system may include an optical energy source, to produce optical energy for optical transduction. In addition, such a system may provide a transmitter to transmit the emitted signals (e.g. as emitted from a fluorescent reacted substrate 35). The detection, processing, and storage of such signals and processing results are known to those skilled in the art and are not discussed herein further. Moreover, the embodiments described herein can be used with any aptamer-based endpoint sensor; even with upstream polymerase chain amplification (mass amplification of nucleic acids) where recognition probe 10 could be readily incorporated into the polymerase chain reaction (PCR) primers which would in-turn produce an amplified, labeled product. Additionally, any enzyme substrate will suffice and there are many commercially available options.

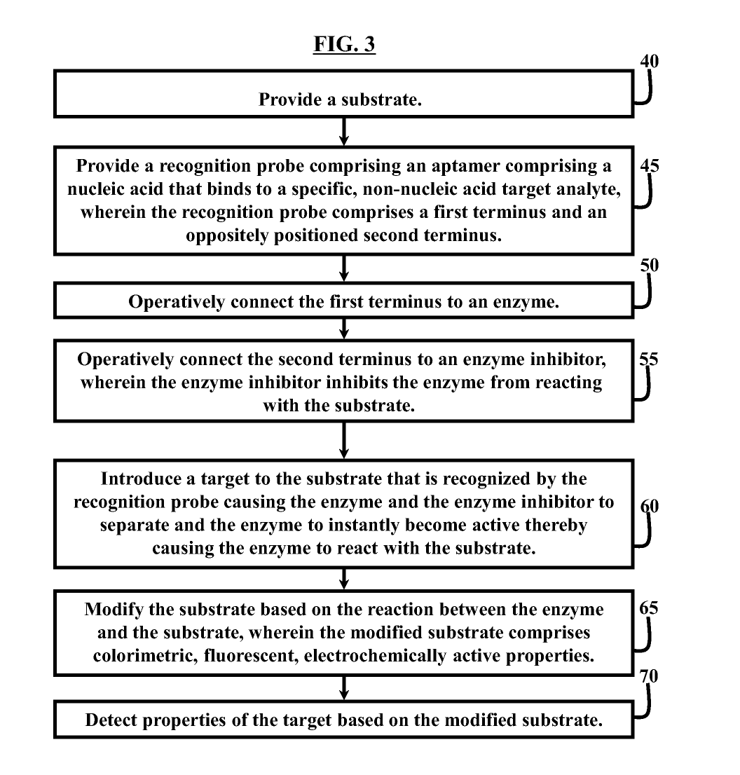

FIG. 3, with reference to FIGS. 1 and 2, is a flow diagram illustrating a method according to an embodiment herein. As shown in FIG. 3, step 40 describes providing a substrate (e.g., substrate 25). Step 45 describes providing a recognition probe (e.g., recognition probe 10) comprising an aptamer comprising a nucleic acid that binds to a specific, non-nucleic acid target analyte (e.g., target 30), wherein the recognition probe comprises a first terminus (e.g., terminus 11) and an oppositely positioned second terminus (e.g., terminus 17). Step 50 describes operatively connecting the first terminus (e.g., terminus 11) to an enzyme (e.g., enzyme 15, via enzyme attachment 13). Step 55 describes operatively connecting the second terminus (e.g., terminus 17) to an enzyme inhibitor (e.g., enzyme inhibitor 20, via enzyme inhibitor attachment 14), wherein the enzyme inhibitor inhibits the enzyme from reacting with the substrate. Next, step 60 describes introducing a target (e.g., target 30) to the substrate (e.g., substrate 25) that is recognized by the recognition probe (e.g., recognition probe 10) causing the enzyme (e.g., enzyme 15) and the enzyme inhibitor (e.g., enzyme inhibitor 20) to separate and the enzyme to instantly become active thereby causing the enzyme to react with the substrate. Step 65 describes modifying the substrate (e.g., reacted substrate 35) based on the reaction between the enzyme and the substrate, wherein the modified substrate (e.g., reacted substrate 35) comprises any of colorimetric, fluorescent, and electrochemically active properties. For example, modification of the substrate may include, but is not limited to, precipitate change, change in color, the substrate could become luminescent, the substrate could become chemiluminescent, the substrate could become electroactive, or could contain other qualitative, quantitative, measurable, or unmeasureable characteristics. In general, the modified substrate could have other changes that are measurable. Step 70 of FIG. 3 then describes detecting properties of the target (e.g., target 30) based on the modified substrate (e.g., reacted substrate 35).

The embodiments described herein provide improved sensitivity over immunoassays (e.g., antibody assays without amplification). In addition, the embodiments described herein provide faster and simpler (e.g., less logistics) detection of an analyte when compared to traditional ELISA. Benefits resulting from these embodiments are broad and cover several fields including biological and chemical agent detection and diagnostics and biological and medical diagnostic arrays, as well as pharmaceutical applications such as drug discovery and proteomics.

Thus, embodiments herein provide a novel single-step structure switching probe (e.g., aptamer-based probe 1) combined with an enzymatic signaling (e.g., enzyme 15 and enzyme inhibitor 20) which allows for improved reliability; e.g., lower false alarm rates. The embodiments described herein allow for two independent ON/OFF events, for one binding event which minimizes environmental interference. Thus, a single-step recognition and signaling element employing enzymatic signaling aptamer-based recognition elements is provided and is an improvement of what is currently known in the art. Moreover, those of skill in the art will appreciate that other recognition element systems are possible in accordance with the embodiments herein. For example, the incorporation of another recognition element, such as a Peptide Nucleic Acid (PNA) or another element that exhibits a conformational change upon binding can be attached to an enzyme-enzyme inhibitor pairing to provide a method, test, or device that would detect the presence of a biological species as described herein.

Chemical and biological sensors should ideally have the analytical characteristics of high sensitivity (low detection limits), reproducibility, and reliable (meaning low false positives and negatives) and speed. Furthermore, the embodiments described herein address reliability problems by introducing complementary enzyme/enzyme inhibitor in a single-step approach. By incorporating both an enzyme and an enzyme inhibitor with an aptamer having a stem-loop, embodiments described herein provide added reliability for both improved false alarm and false negative rates. Furthermore, embodiments described herein improve speed and provide for low logistics.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An aptamer probe system comprising:
an enzyme substrate compound;
an aptamer recognizing an analyte; and
a recognition probe comprising:
a first terminus operatively coupled to an enzyme catalyzing said enzyme substrate compound;
a second terminus operatively coupled to an enzyme inhibitor corresponding to said enzyme, wherein said aptamer is positioned between said first terminus and said second terminus; and
a stem loop structure positioned between said first terminus and second terminus.

2. The system of claim 1, wherein said enzyme inhibitor prevents said enzyme from catalyzing said enzyme substrate compound.

3. The system of claim 2, wherein, after exposure to an analyte that is a complement to said aptamer, said aptamer is structurally altered to sufficiently separate said enzyme inhibitor and said enzyme to restore said enzyme to catalyzing said enzyme substrate compound.

4. The system of claim 1, wherein said recognition probe comprises an anti-thrombin aptamer.

5. The system of claim 1, wherein said recognition probe forms a G-quartet in the presence of a thrombin protein.

6. The system of claim 1, wherein said analyte comprises any of a protein, a peptide, a peptide nucleic acid, a nucleoside triphosphate, a carbohydrate, a lipid, a virus, a cell fragment, and a whole cell.

7. The system of claim 1, wherein said enzyme comprises any of a nuclease, a protease, and a glycosidase.

8. The system of claim 1, wherein said enzyme comprises a hydrolase enzyme.

9. The system of claim 1, wherein said enzyme comprises a butyrylcholinesterase and wherein said analyte comprises a cholinesterase inhibitor.

10. The system of claim 1, wherein said enzyme substrate compound comprises any of acetylcholine and butyrylcholine, and wherein said enzyme comprises any of acetylcholinesterase and butyrylcholinesterase.

11. The system of claim 9, wherein said enzyme substrate compound comprises benzoyl-arginine-ethyl-ester, and wherein said enzyme comprises papain.

12. The system of claim 1, wherein said enzyme substrate compound comprises urea, and wherein said enzyme comprises urea aminohydrolase.

13. The system of claim 1, wherein said stem loop structure comprises a stem comprising a double-stranded region having a length that is greater than six nucleotides.

14. The system of claim 12, wherein said enzyme inhibitor comprises a small-molecular phosphoramidite.

15. The system of claim 12, wherein said enzyme substrate compound comprises DABCYL-bAla-ala-Gly-Leu-AlaBAla-EDANDS.

16. An aptamer probe apparatus comprising:
an aptamer;
a recognition probe comprising:
a first terminus coupled to an enzyme; and
a second terminus coupled to an enzyme inhibitor,
wherein said aptamer is positioned between said first terminus and said second terminus;
an enzyme substrate compound that becomes any of colorimetric, fluorescent, and electrochemically active when catalyzed by said enzyme; and
a structure incorporated into said recognition probe that brings said first terminus and said second terminus within close proximity to each other, wherein said enzyme inhibitor prevents said enzyme from catalyzing said enzyme substrate compound.

17. The apparatus of claim 16, wherein, after exposure to an analyte that is a complement to said aptamer, said aptamer is structurally altered to sufficiently separate said enzyme inhibitor and said enzyme to restore said enzyme to catalyzing said enzyme substrate compound.

18. The apparatus of claim 16, wherein said enzyme inhibitor comprises a small-molecular phosphoramidite.

19. The apparatus of claim 16, wherein said enzyme substrate compound comprises DABCYL-bAla-ala-Gly-Leu-AlaBAla-EDANDS.

20. An aptamer probe system comprising:
an enzyme substrate compound;
an aptamer complementing an analyte; and
a recognition probe comprising a first terminus operatively coupled to an enzyme catalyzing said enzyme substrate compound and a second terminus operatively coupled to an enzyme inhibitor corresponding to said enzyme,
wherein said aptamer is positioned between said first terminus and said second terminus and forms a structure where said enzyme inhibitor, coupled to said second terminus, interacts with said enzyme, coupled to said enzyme to thereby inhibit said enzyme catalyzing said enzyme substrate compound.

21. A method of detection, said method comprising:
providing a substrate;
providing a recognition probe comprising an aptamer comprising a nucleic acid that binds to a specific, non-nucleic acid target analyte, wherein said recognition probe comprises a first terminus and an oppositely positioned second terminus;
operatively connecting said first terminus to an enzyme;
operatively connecting said second terminus to an enzyme inhibitor, wherein said enzyme inhibitor inhibits said enzyme from reacting with said substrate;
introducing a target to said substrate that is recognized by said recognition probe causing said enzyme and said enzyme inhibitor to separate and said enzyme to instantly become active thereby causing said enzyme to react with said substrate;
modifying said substrate based on a reaction between said enzyme and said substrate, wherein said modified substrate comprises any of colorimetric, fluorescent, and electrochemically active properties; and
detecting properties of said target based on said modified substrate.
